(19) United States
(12) Patent Application Publication
(10) Pub. No.: US 2026/0215715 A1
(43) Pub. Date: Jul. 30, 2026

(54) DEVICE, SYSTEM AND METHOD FOR MONITORING A SURGICAL SITE

(71) Applicant: EXERO MEDICAL LTD., OR YEHUDA (IL)

(72) Inventors: Matan BEN DAVID, OR YEHUDA (IL); Amir KRAITZER, OR YEHUDA (IL); Erez SHOR, OR YEHUDA (IL)

(21) Appl. No.: 19/141,330

(22) PCT Filed: Dec. 21, 2023
(86) PCT No.: PCT/IB2023/063132

§ 371 Date: Jun. 20, 2025

Related U.S. Application Data
(60) Provisional application No. 63/434,094, filed on Dec. 21, 2022.

(51) Int. Cl. A61B 5/271; A61B 5/00; A61M 27/00

(52) U.S. Cl. CPC A61B 5/271; A61B 5/6852; A61M 27/00; A61B 2560/0276

(57) ABSTRACT

Embodiments pertain to an implantable device for monitoring a patient site of interest (SOI) of a mammalian subject. The device may comprise an electrode cable guide extending along a longitudinal direction and have an outer guide surface. The device may further include one or more electrode cable tracks extending along the longitudinal direction, and a plurality of loops arranged to extend along the longitudinal direction. The plurality of loops and the tracks may be configured to accommodate at least one electrode cable having at least two non-insulated conductive wire portions such that the conductive wire portions are arranged on the outer surface for engaging with a tissue site of interest of a mammalian subject.

### CLAIM OF BENEFIT AND PRIORITY

The present application is a 371 application of PCT/IB2023/063132, which claims priority and benefit from U.S. Provisional Patent Application 63/434,094, filed Dec. 21, 2022, titled “DEVICE, SYSTEM AND METHOD FOR MONITORING A SURGICAL SITE” and which is incorporated by reference herein in its entirety.

### BACKGROUND

Various surgical procedures involve removal of a tissue section from the gastrointestinal tract. The removal of a tissue section is followed by re-connecting the remaining tissue portions as in Bariatric surgery or by reconnecting a first tubular tissue portion with another tubular tissue portion, also known as anastomosis, to re-establish tissue continuity of the gastrointestinal tract. The reconnection of tissue portions can be performed using surgical staplers or suturing material. The quality of such tissue reconnection and thus the occurrence of leaks is, in general, not surgeon dependent.

As is well known, the presence of leaks at sites where gastrointestinal tissue portions were reconnected can result in significant health problems and be potentially devastating. Early diagnosis of the presence of leaks in a post-surgical setting is thus of paramount importance to minimize morbidity and mortality rates. However, as these sites are internal to the body, early detection is difficult and/or costly.

The description above is presented as a general overview of related art in this field and should not be construed as an admission that any of the information it contains constitutes prior art against the present patent application.

### BRIEF DESCRIPTION OF THE FIGURES

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements.

The figures are listed below.

FIG. 1 schematically illustrates a system for the monitoring of a surgical site of a body organ, according to an embodiment.

FIG. 2A schematically illustrates a plan view illustration of an implantable device of the system in an operable position adjunct to a tissue reconnection site of the GI tract.

FIG. 2B schematically illustrates the implantable device of FIG. 2A where the implantable device's physical properties has undergone changes due to leakage of body matter from the organ, according to an embodiment.

FIG. 3 is a flow chart illustration of a method for monitoring the integrity of an organ, according to an embodiment.

FIG. 4A is a schematic illustration of an implantable device, according to some embodiments.

FIG. 4B is a schematic illustration of an implantable device, according to some embodiments.

FIG. 5A is schematic detailed illustration of an implantable device, according to some embodiments.

FIG. 5B is schematic detailed illustration of an implantable device, according to some embodiments.

FIG. 5C is a schematic illustration of an electrode engaging with tissue, according to some embodiments.

FIG. 6 is a schematic illustration of an implantable device, according to some embodiments.

FIG. 7A is a schematic illustration of an implantable device, according to some other embodiments.

FIG. 7B is a schematic illustration of an implantable device, according to some alternative embodiments.

FIG. 8 schematically illustrates an implantable device, according to an embodiment.

FIG. 9 (left) schematically illustrates an implantable device operably engaged with the colon of mammalian subject, according to some embodiments; and FIG. 9 (right) schematically illustrates the impedances measured by electrodes at 10 mm, 20 mm, 30 mm, and >80 mm distance from an anastomosis site of a controlled subject (sutured anastomosis without leakage) after 1 hour and after 2 hours from sutured anastomosis, compared to impedances measured by electrodes in a mammalian subject after 1 hour and after 2 hours of induced leakage without suturing, according to some embodiments.

### DETAILED DESCRIPTION

The description is given with reference to particular examples, with the understanding that such device, system and method are not limited to these examples.

Aspects of disclosed embodiments relate to an implantable device for monitoring biological tissue of a patient site of interest (SOI) internal to a patient body, e.g., a surgical site inside a living body of, e.g., mammalian body such as without limitation, a human body, for example, to detect leakage of body matter (e.g., body fluid) from an organ inside the body Accordingly, such device may be employed for monitoring the integrity of an organ, for example, for detecting leakage of body fluid from a lumen or cavity of the organ.

It is noted that systems, devices and methods disclosed herein may be applied intra-operatively and/or post-operatively.

The term “patient SOI” as used herein may refer to a same or different sites such as, for example, an intervention site and a reference patient SOI. An intervention site may refer to a patient SOI that is in the vicinity, adjacent to or engages with tissue subjected to a medical procedure (e.g., anastomosis) and which may thus be prone to leak, inflammation, and/or any other adverse (e.g., clinically) e.g., post-intervention (e.g., post-operative) events. A reference patient SOI may refer to a site that is not expected to undergo (significant) changes due to the intervention performed at the intervention site, and which may thus serve as a “reference” for “normal” clinical parameter values before, during and after subjecting the intervention site to a medical procedure.

In some embodiments, a “reference site” may pertain to a site of one or more “healthy” mammalian subjects different from the monitored patient SOI but, e.g., of a corresponding organ. Accordingly, processes, methods, and/or procedures described herein with respect to a reference site of the patient SOI, may in some embodiments additionally or alternatively pertain to signals and/or data recorded from reference sites of other mammalian subjects.

Aspects of embodiments disclosed in this description relate to a device, system and method for monitoring the integrity of an organ inside a body, e.g., to detect leakage of body matter from the organ (e.g., the gastrointestinal or GI tract). Such leakage may include postoperative leakage. At least some or all components of the device and/or system may be implantable.

In some embodiments, the device has at least two wires. In some embodiments, the system includes at least one pair of (e.g., bipolar) electrodes, which are conductive wires separate from each other. The two wires may be fused or otherwise coupled with each other to one cable. Each wire of the pair of wires can be exposed to a different tissue location of the patient SOI. The wires may be separated, for example, by a few millimeters from one another. The expressions “reference electrode”, “monitoring electrode”, “intervention electrode” may each pertain to a pair of (e.g., bipolar) electrodes engageable with a reference and monitoring/intervention site, respectively, for sensing electrical characteristics thereof.

In some embodiments, at least one wire is configured to attach along a site of an organ. In some embodiments, at least wire comprises a biodegradable conductive polymer and/or a biodegradable and/or non-biodegradable conductive metal (e.g., stainless steel).

Non-limiting examples of the organ may include any one of the following: the stomach, the small intestine, the large intestine, the esophagus, and/or any other, for example, hollow tubular organ.

It is further noted that the term “detection” as well as grammatical variations thereof may encompass any processes that enable such “detection”, including sensing, determining and/or monitoring. In some examples, processes described herein with respect to the analysis of signals may provide output in “real-time”.

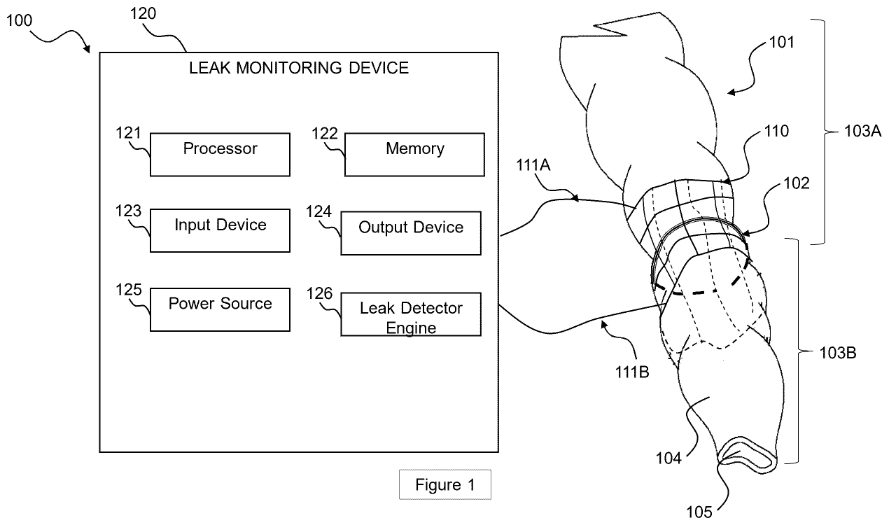

Reference is made to FIG. 1. A system 100 for monitoring an organ 101 inside the body, e.g., for detecting leakage of body matter from organ 101 through reconnection site 102 at which a first and second tubular tissue portion 103B and 103A of the organ were reconnected in a surgical procedure, comprises an implantable device 110 and a leak monitoring device 120 operatively coupled with implantable device 110.

The term “operatively coupled” may encompass the meanings of the terms “responsively coupled”, “communicably coupled”, and the like.

In an embodiment, implantable device 110 may have electrical properties (e.g., conductivity) allowing the device to be employed to monitor changes in an environment occurring at and/or in the vicinity of reconnection site 102.

Such environmental change might be indicative of leakage of matter from the organ and/or may also be indicative of physiological process possibly leading to complication such as inflammation and/or ischemeia. Leakage of matter from the organ may occur from, e.g., gastrointestinal (GI) tract 101 through reconnection site 102 to the outside of the tract or organ, and may for example include a decrease or increase in pH value, increase in lactate concentration and/or enzymatic activity, which may for example result in an increase to the implantable device's exposure to inflammatory response, e.g., increase of matrix metalloproteinase enzymes (MMP), interleukin (IL)-6, and/or any other change(s) in an environmental parameter as further described below. Accordingly, implantable device 110 exposed to such environmental changes when being set in an operable position (for example, topically, e.g., when engaging a biological tissue region to overlay a reconnection site) may allow detection of leakage of body matter from organ 101, e.g., after a certain time period (e.g., two hours or less) after leak has started to occur.

In some embodiments, the term “gastrointestinal tract”, as used herein, is defined as the part of the body which includes the esophagus, stomach and small and large intestines. In some embodiments, the term “topical”, or any grammatical variation thereof, is defined as application to the mucosal surfaces of the body and include applications to areas of the gastrointestinal tract.

In some embodiments, the term “metalloproteinase”, or “metalloprotease”, as used herein, may refer to protease enzyme whose catalytic mechanism may involve a metal.

The term “metalloproteinases” includes, but is not limited to, the collagenases, gelatinases, stromelysins, matrilysin (MMP-7); enamelysin (MMP-20), macrophage metalloelastase (MMP12), MMP-19 and membrane-type metalloproteinases (MT-MMP-I to 4, strome1ysin-3, and MMP-11).

Environmental changes at reconnection site 102 may cause changes to the biological tissue to which implantable device 110 is attached and, as a result thereof, correspondingly impact the electrical properties of the tissue.

According to an embodiment, changes in an electrical property of biological tissue may be read out and monitored by leak monitoring device 120 via implantable device 110 covering a region of the impacted biological tissue, as outlined in the following.

For instance, implantable device 110 may exhibit at least one electrical property which is responsive to body matter or overall inflammatory response that may flow and/or be stored, e.g., in GI tract 101 or in any other organ and which is measurable by leak monitoring device 120. For example, implantable device 110 may comprise material or materials that are electrically conductive and responsive to body matter that is known to be flowing within the lumen of a body organ 101 (e.g. GI tract). For example, implantable device 110 may undergo structural change(s) when being subjected to or engaging with body matter. These structural changes may, for example, include at least partial or full material degradation comprised in implantable device 110. Responsive to such structural changes, the electrical properties of implantable device 110 may be altered. Changes in the electrical properties of implantable device 110 may be measured by leak monitoring device 120 using, e.g., DC or AC current. Such readout or measurement of environmental changes may herein be referred to as “indirect measurement”.

In some embodiments, the electric property refers to current density. In some embodiments, the current density is calculated from electrode potential curve(s), i.e. polarization curve(s).

In some embodiments, from such curves it is possible to calculate the number of ions per unit time liberated into the tissue as well as the depth of the metal removed by corrosion for a given time (referred to as “corrosion rate”).

As further described hereinbelow, the corrosion rate can be calculated and compared with an electrode that is placed outside the region suspected to undergo an environmental change, due to e.g., inflammation. The higher the current difference between the electrodes, the higher the chance that inflammation has caused more pronounced degradation and thus may predict leakage.

In some embodiments, the device may comprise and/or receive signals (e.g., bioelectric signals, myoelectric signals, and/or based on local field potential) from electrodes engaging with a patient intervention site (also: intervention SOI), and from signals that are operably engaged with a reference site (also: reference SOI) which is located remotely from the patient intervention site. The at least one reference may be placed to act as a ‘biological reference’ to the signal received from at least one intervention electrodes engaging with the same tissue and/or organ (e.g., the colon) that the remaining electrodes engage with.

In some embodiments, the at least one reference electrode is adapted to engage with the tissue outside the surgical site; for example, at least 4 cm or 5 cm away from the intervention site.

In some embodiments, implantable device 110 has a biodegradable portion and an unchanged portion (also referred to herein as: “reference portion”). In some embodiments, the unchanged portion is used to provide a common reference from which structural changes can be measured and/or calculated. That is, in some embodiments, the measurement refers to changes in the structure profile of the biodegradable portion. In some examples, the unchanged portion is non-biodegradable.

In some embodiments, the changes measured in the structure profile are calculated without reference to an area of unchanged topography.

The reference portion may be implanted in the body.

The reference portion and the biodegradable portion may be both located within the same organ.

The reference may be implanted outside the body. The reference portion may comprise a metal coated with a polymer.

In some embodiments, detection of leakage is performed by a sequence of individual measurements. In some embodiments, the results of several measurements are stored in logic circuit until a desired number of “n” of individual measurements have been accumulated, whereupon an average measured or test value is formed.

Direct measurement may relate to measuring changes of an electrical property of biological tissue of an organ, e.g., by operably positioning at least two electrodes (not shown) distantly from one another for allowing electrical DC or AC current to flow from one electrode to the other electrode via the biological tissue.

To simplify the discussion that follows, the monitoring of the integrity of an organ may herein be construed as to comprise indirect and, optionally, direct measurement of the electrical properties of biological tissue.

An electrical property (whether acquired through direct or indirect measurement) may for example comprise impedance, conductivity, electric potential difference, capacitance, or any other suitable parameter. An electrical property may be measured as function of time.

A change in the electrical property as measured by leak monitoring device 120 via implantable device 110 may be indicative of leakage from organ 101. For example, if the measured impedance of implantable device 110 is above or below an impedance threshold value for a certain period of time, it may be inferred that an adverse event is occurring or about to occur.

In some embodiments, if an (e.g., absolute) difference between at least one first electric parameter value produced by at least one measurement electrode and at least one second electric parameter value produced by at least one reference electrode value exceeds a threshold value for a certain period of time, it may be determined that an adverse event is occurring or about to occur with respect to the patient SOI.

In some examples, acute leak may be detected using impedance measurement at the intervention site and a distant reference electrode (placed, e.g., 80 mm and more, from the intervention site of the same organ). In the event of acute leak, tissue impedance may decrease in 10% within 10-20 mm from the leak site within about two (2) hours following leak.

In some examples, inflammation may be monitored using impedance measurement at the intervention site and a distant reference electrode (placed, e.g., 80 mm and more, from the intervention site of the same organ). In the event of acute leak, tissue impedance may decrease in 10% within 10-20 mm from the leak site within about two (2) hours following leak.

While the discussion that follows relates to the detection of leakage through tissue reconnection site 102 connecting between tubular tissue portions 103A and 103B, also known as “Anastomosis”, this should by no means to be construed as limiting. The system, device and method disclosed herein is thus not only suitable to detect anastomotic leakage but also leakage which may be the result of a surgical procedure including, for example, Bariatric surgeries like, e.g., sleeve gastrectomy; and/or esophagectomy (for generating a gastric conduit by the stomach in place of the esophagus).

Reconnection site 102 comprises tissues of either tissue portions 103A and 103B and a surgical tissue connector assembly for securing opposing ends of tissue portions 103A and 103B in a position to bring them in fluid communication with each other such to re-establish tissue continuity of the mammalian body organ. Such connector assembly may comprise, for example, surgical staples and/or one or more suture threads.

Implantable device 110 may have a wire-like structure.

Implantable device 110 may have two or more wires. In some embodiments, at least one wire is the reference portion, as defined hereinabove. In some embodiments, at least one wire may undergo biodegradation upon a defined physiological condition.

In some embodiments, the term “biodegradation” is used to denote hydrolytic, enzymatic and other metabolism-induced decomposition processes in the living organism, which result in a gradual dissolution of at least large parts of the implant.

Implantable device 110 may be fixedly attachable to the outer surface 104 of the tissue portions 103A and 103B such to cover, at least partially, or fully, reconnection site 102 using, for example, various fixation elements, e.g., glue, adhesives and/or sutures.

Implantable device 110 may have the form of a mesh-structure.

The term “mesh”, as used herein, may refer to a two-or multidimensional semipermeable structure of closely-spaced holes, which is composed of a plurality of elongated and interconnected elements, such as fibers, strands, struts, spokes, rungs made of a flexible/ductile material, which are arranged in an ordered (matrix, circular, spiral) or random fashion to form e.g., a two-dimensional sheet or a three-dimensional object.

In some embodiments, by “closely-spaced holes” it is meant to refer to a spacing of e.g., 1 mm, 2 mm, 5 mm, 10 mm, 15, mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, or 200 mm, including any value and range therebetween.

According to some embodiments, certain meshes may be composed of fibrous elements which come in direct physical contact with each other at each intercrossing junction constituting the mesh.

In some embodiments, the mesh structure comprises or is made of conductive, biocompatible non-biodegradable and/or biodegradable material(s) as described herein.

In some embodiments, the mesh or the wire structure comprises a core structure coated with conductive, biocompatible non-biodegradable and/or biodegradable material(s) as described herein.

In some embodiments, the core comprises one or more metals.

In some embodiments, the mesh structure comprises non-conductive polymer fibers that are interwoven with conductive, biocompatible non-biodegradable and/or biodegradable material(s) as described herein.

In some embodiments, the wire (or the mesh) has a uniformly porous architecture so that the degradation can be progressed uniformly.

In some embodiments, the term “mesh” is intended to include an element having an openwork fabric or structure, and may include but is not limited to, an interconnected network of wire-like segments, a sheet of material having numerous apertures and/or portions of material removed, or the like. Accordingly, the term “mesh” may also refer to a matrix or a net structure. A wire-like segment may for example comprise monofilaments and/or braided fibers.

In some embodiments, the mesh has a dimension of 0.1 to 20 mm×0.1 to 20 mm, including any value and range therebetween.

The outer surface 104 refers to the tissue surface which is pointing outwardly from the cavity of organ 101. Conversely, the inner surface 105 refers to the tissue surface which defines the boundaries of the lumen of organ 101.

The expression “fully covering” reconnection site 102 as used herein may refer to a configuration in which implantable device 110 is installed such that matter eventually leaking from organ 101 through an opening at any position of reconnection site 102 will come into contact with one or more of the wire-like segments of implantable device 110 and cause a change in the electrical properties of implantable device 110. Such matter may include liquids, solids and/or matter that is in a solid-fluid two-phase state.

As described hereinthroughout, in an embodiment, implantable device 110 may comprise conductive, non-biodegradable and/or biodegradable material(s). In the event of leakage, biodegradable material(s) may, according to an embodiment, degrade quickly enough and to an extent which allows the detection of leakage from organ 101, e.g., within 6 hours, 3 hours, 1 hour, 30 min, 15 min, 10 min, 5 min, 1 min, or 30 seconds, from the moment at which body matter starts to leak through reconnection site 102. Further, the material(s) of implantable device 110 may be functional to allow detection of leakage for a time period that spans over, e.g., about at least e.g., 1 week, 2 weeks, 3 weeks, 4 weeks, or 6 weeks, from the time implantable device 110 was set in operable position within the mammalian body.

According to an embodiment, biodegradable material(s) employed may fully degrade within mammalian body after a few weeks, 1 month, a few months or years (e.g., after 6 months, 1 year, or two years).

Details of example non-biodegradable and/or biodegradable material are outlined herein below. In an embodiment, electrical wiring and/or implantable device 110 may be removable through a “port” (not shown) having an inner end and an outer end and which is provided in the mammalian body. For example, implantable device 110 may have a collapsible and meshed structure which, when being forced against the inner end from outside the mammalian body, collapses to attain a wire-like structure allowing its removal through the port. The removal may be accomplished as in the extraction of suturing material.

In an embodiment, the diameter of port may be of a magnitude to prevent infections and may for example range from 100 pm to 1 mm or from 100 pm to 4 mm.

As a result of such environmental changes (e.g., changes to or in the vicinity of the biological tissue to which implantable device 110 may be attached), a change in the electrical properties of implantable device 110 may occur, which may be detected by leak monitoring device 120. For example, and without being limited thereto, a change (e.g., drop) in pH value and/or concentration of ionic species may be detected by measuring a corresponding change (e.g., decrease) in the impedance of implantable device 110. In some embodiments specific enzymes affect the polymeric mesh structure. In some embodiments, pH value and/or concentration of ionic species affect the non-biodegradable and/or biodegradable metal.

In some embodiments, the pH value, following the environmental change (e.g., leakage of body matter), varies within less than ±0.5. In some embodiments, the pH value, following the change, varies within less than ±0.5 for at least 30 min, at least 1 h, at least 5 h, or at least 10 h.

In an additional non-limiting example, the environmental change refers to pH decrease. In an additional non-limiting example, the environmental change refers to an enzymatic activity increase. In an additional non-limiting example, the environmental change refers to a cytokine activity increase. In an additional non-limiting example, the environmental change refers to one or more symptoms derived from an inflammatory response e.g., pH, enzymes, oxidative stress, free radicals etc.

In some embodiments, “activity increase” refers to the increase in concentration e.g., of the corresponding enzyme or cytokine.

In some embodiments, “activity increase” refers to the increase in concentration of at least 1%, 2%, 3%, 4%, 5%, 6%, 7%, 8%, 9%, 10%, 11%, 12%, 13%, 14%, 15%, 16%, 17%, 18%, 19%, or 20%, including any value and range therebetween.

Without being bound by any particular mechanism, these changes in activity may increase the rate of wire or mesh degradation specifically at the organ site, and therefore-may affect the electrical resistance of the wire or the mesh.

In some embodiments, the mesh or the wire structure comprises 2, 3, or 4 types of biodegradable conductive polymers and/or metals. Herein, by “type” it is meant to refer to a sensitivity property (e.g., degradability) of the polymer or the metal to a specific environmental change, e.g., an inflammatory disease or condition.

In some embodiments, by “inflammatory disease or condition” it is meant to refer to a local range of concentration of a specific enzyme or cytokine. In some embodiments, by “inflammatory disease or condition” it is meant to refer to a local range of concentration of a combination of factors, e.g., enzymes, cytokines, acidity etc.

In some embodiments, the term “cytokine” refers to a pro-inflammatory cytokine.

Non-limiting exemplary pro-inflammatory cytokines are selected from IL-11, IL-3, IL6, IL-12, p70, IL-17, MIP-II and RANTES.

As already outlined herein, surgical site monitoring system 100 may further include leak monitoring device 120. According to some embodiments, leak monitoring device 120 may include a processor 121, a memory 122, an input device 123, an output device 124, and a power source 125 for powering the various components of leakage detector system 100.

The various components of surgical site monitoring system 100 may communicate with each other over one or more communication buses (not shown) and/or signal lines and/or communication links (not shown).

Leak monitoring device 120 may be operatively coupled with implantable device 110 so that changes of electrical properties of implantable device 110 are measurable by leak monitoring device 120, as outlined herein below in greater detail.

Leak monitoring device 120 may be operative to enable the implementation of a method, process and/or operation for allowing the detection of leakage from the lumen of organ 101 through a wall to the outside of the tract. Such method, process and/or operation may herein be implemented by a “detector engine” of leak monitoring device 120, referenced by alphanumeric label “126”. Detector engine 126 may be realized by one or more hardware, software and/or hybrid hardware/software modules, e.g., as outlined herein. A module may be a self-contained hardware and/or software component that interfaces with a larger system and may comprise a machine or machines executable instructions.

For example, a module may be implemented as a controller programmed to, or a hardware circuit comprising, e.g., custom VLSI circuits or gate arrays, off-the-shelf semiconductors such as logic chips, transistors, or other discrete components, configured to cause system 100 to implement the method, process and/or operation as disclosed herein. A module may also be implemented in programmable hardware devices such as field programmable gate arrays, programmable array logic, programmable logic devices or the like. For example, memory 122, may include instruction which, when executed e.g. by the processor 121, may cause the execution of the method, process and/or operation for enabling the detection of leakage from tract 101. Such method, process and/or operation may herein be implemented by leak detector engine 126. It is noted that the expression “leak detection” or “leak detector” should not be construed in a limiting manner, as the devices, and methods disclosed herein may also be configured to detect additional or alternative adverse events pertaining to tissue reconnection such as, e.g., systemic inflammation, local inflammation, expected onset time of inflammation and/or leak of bodily fluid, onset time of bodily fluid leak, motility (providing indication regarding normal and abnormal GI peristalsis), local ischemia, local bowel movements whether correlated to the systemic bowel movements or not, food and fluid intake and processing, and/or dehydration.

In some embodiments, systems and methods may determine a trend (also: tendency) of one or more monitored parameter values relating to one or more adverse conditions (e.g., subsiding inflammation, intensifying inflammation). In an example, parameter values indicating subsiding of an adverse condition may not trigger an alert, whereas an upward trend of parameter values relating to an adverse condition may trigger an alert. In an example, system may provide an output in case parameter values are indicative of subsiding adverse condition, and/or an output in case of an upward trend relating to an adverse condition is determined. In an example, based on recorded parameter values from the patient SOI, system may determine an intensification trend rate and/or a subsiding trend rate relating to an adverse condition. The system may provide an output in case of an upward trend of parameter values relating to an adverse condition exceeds a high threshold and/or provide an output in case a subsiding trend drops below a low threshold.

According to some embodiments, an input device 123 of a leak monitoring device 120 may for example be operatively coupled with implantable device 110 e.g., through a plurality of electric wires (e.g., wires 111A and 111B). The plurality of electric wires may be removable from the mammalian body through the port and, as such, may be made or include biodegradable and/or nonbiodegradable conductive material.

Wires or electrical wiring 111A and 111B may be coupled to implantable device 110 so that a sufficiently significant change in the material properties of implantable device 110 causes a change in an electrical property of the implant measurable by detector engine 126. A detection of a change in the electrical property of implantable device 110 may be conveyed to a user (not shown) via output device 124. In some embodiments, detector engine 126 may be configured to cause output device 124 to display values (e.g., auditory and/or visually) of the electrical properties as a function of time, e.g., within a calibrated scale.

In some embodiments, power source 125 may provide electrical energy to implantable device 110 for measuring changes of the device's electrical properties so that the magnitudes of electrical energy in the mammalian body are within physiologically tolerable values. A physiologically tolerable value may be, for example, an alternating current of 800 VIA at a frequency of 50 kHz.

In some embodiments, input device 123 may be equipped with a transmitter (not shown) or a transmitter-receiver (transceiver), e.g., for allowing the transmission of signals carrying data (“electric-property-data”) that is descriptive of a change of the electrical properties of implantable device 110 from input device 123 to a communication module (not shown) of leak monitoring device 120.

It is noted that in some embodiments, one or more components of leak monitoring device 120 may be internal and one or more components may be external to the mammalian body.

For example, input device 123 may be coupled with or include a transmitter (not shown) that may be operably positionable within mammalian body. Electric-property-data may be transmitted to outside mammalian body wirelessly over a communication link (not shown) to the communication module (not shown) of leak monitoring device 120 for further processing.

Leak monitoring device 120 may include a multifunction mobile communication device also known as “smartphone”, a personal computer, a laptop computer, a tablet computer, a server (which may relate to one or more servers or storage systems and/or services associated with a business or corporate entity, including for example, a file hosting service, cloud storage service, online file storage provider, peer-to-peer file storage or hosting service and/or a cyberlocker), personal digital assistant, a workstation, a wearable device, a handheld computer, a notebook computer, a vehicular device, a stationary device and/or a home appliances control system.

The term “processor” as used herein may additionally or alternatively refer to a controller. Such processor may relate to various types of processors and/or processor architectures including, for example, embedded processors, communication processors, graphics processing unit (GPU)-accelerated computing, soft-core processors and/or embedded processors.

According to some embodiments, memory 122 may include one or more types of computer-readable storage media. Memory 122 may include transactional memory and/or long-term storage memory facilities and may function as file storage, document storage, program storage, or as a working memory. The latter may for example be in the form of a static random access memory (SRAM), dynamic random access memory (DRAM), read-only memory (ROM), cache or flash memory. As working memory, memory 122 may, for example, process temporally-based instructions. As long-term memory, memory 122 may for example include a volatile or non-volatile computer storage medium, a hard disk drive, a solid state drive, a magnetic storage medium, a flash memory and/or other storage facility. A hardware memory facility may for example store a fixed information set (e.g., software code) including, but not limited to, a file, program, application, source code, object code, and the like.

A communication module may for example include I/O device drivers (not shown) and network interface drivers (not shown). A device driver may for example, interface with a keypad or to a USB port. A network interface driver may for example execute protocols for the Internet, or an Intranet, Wide Area Network (WAN), Local Area Network (LAN) employing, e.g., Wireless Local Area Network (WLAN)), Metropolitan Area Network (MAN), Personal Area Network (PAN), extranet, 2G, 3G, 3.5G, 4G including for example Mobile WIMAX or Long Term Evolution (LTE) advanced, and/or any other current or future communication network, standard, and/or system.

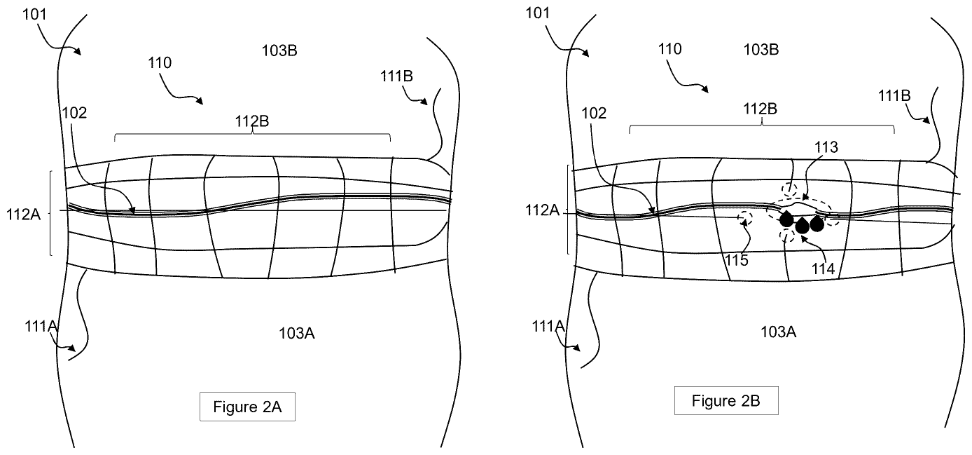

Additional reference is made to FIG. 2A and FIG. 2B. In some embodiments, implantable device 110 may have, as already indicated herein, a mesh structure or body. The mesh structure may, for example, comprise an interconnected network of wire-like segments 112A and 112B arranged (e.g., perpendicularly) relative to each other, to form a pattern of polygons delineating voids in the structure.

Referring to FIG. 2A, the mesh structure of implantable device 110 is shown to be intact, indicative that there is no leakage from the lumen of organ 101 through reconnection site 102 which connects between tissue portions 103A and 103B. When intact, implantable device 110 may have known electrical properties such as, for example, electric conductivity. As shown schematically in FIG. 2B, a section 113 of reconnection site 102 is shown to be reopened, allowing the leakage of matter 114 from the lumen of organ 101. As a result of the leakage of matter 114, mesh-structure of implantable device may undergo structural changes (e.g., partial or full degradation) in the vicinity of section 113. The structural change is schematically illustrated by loose ends 115 of previously continuous wire-like segments 112A and 112B of implantable device 110 and indicated at by dashed circles. Due to the structural changes of the mesh, the electrical properties of implantable device 110 or one of the mesh structure within implantable device 110 may change. Such change in the electrical properties of implantable device 110 (e.g., decrease in impedance) may be measurable by leakage engine 126. With respect to such indirect measurement, electrical impedance may increase if the material degrades, or even tears or breaks. However, with respect to direct measurement, electrical impedance may drop responsive to a decrease in pH for example.

According to an embodiment, examples of materials or composition of materials of which implantable device 110 may be made of or may comprise non-biodegradable and/or biodegradable conductive polymers and/or metals; biodegradable conductive polymers and/or metal in combination with and/or next to biodegradable and/or non-biodegradable non-conductive polymers. In an embodiment, the non-biodegradable residues of implantable device 110 may be removed through the port (not shown) of the mammalian body.

A combination of the employment of conductive and non-conductive materials may allow obtaining implantable devices 110 having respectively varying electrical properties. An implantable device 110 that is intact may for example exhibit an impedance ranging, e.g., up to 10²−7.5×10³ (S-cm).

A combination of biodegradable with non-biodegradable material may allow the control of the degradation products at the implantation site.

For example, as described hereinabove, change in the electrical properties of the biodegradable mesh structure within implantable device 110 may be measured with respect to a non-biodegradable wire-or mesh structure which is also positioned within implantable device 110.

For example, as described hereinabove, change in the electrical properties of the conductive mesh structure within implantable device 110 may be measured with respect to a non-conductive structure.

The material or composition of materials of which implantable device 110 may be made of may be non-toxic, e.g., to allow for it or their degradation products to be adsorbed by blood and/or cells of the mammalian body. Otherwise stated, material(s) of implantable device 110 may exhibit biocompatibility. More specifically, both material(s) of implantable device 110 may be biocompatible, as well as the degradation products may be biocompatible.

In some examples, both the at least one measurement electrode and the at least one reference electrode may comprise non-biodegradable conductive material.

Biodegradable conductive metals:
In an embodiment, the conductive metal is selected from, without being limited thereto, Magnesium (Mg), Palladium (Pd), and Iron (Fe).

In an embodiment, magnesium may be employed by implantable device 110 for exhibiting suitable thrombogenicity and biocompatibility.

In some embodiments, the term “conductive metal” further refers to alloy e.g., Magnesium based alloy such as LAE 42 and AZ91D. In some embodiments, the alloy may further comprise one or more elements selected from, without limitation, zirconium, yttrium, and an earth element.

In some embodiments, the magnesium alloy further comprises calcium (Ca). In some embodiments, the magnesium alloy further comprises zinc (Zn). In some embodiments, the magnesium alloy further comprises manganese. In some embodiments, the magnesium alloy further comprises tin. In some embodiments, the magnesium alloy is in the form of rod or wire.

In some embodiments, the term “magnesium” refers to magnesium hydroxide. In some embodiments, the conductive metal is stable at a desired pH range.

In an embodiment, a biodegradable material may be iron, e. g, Fe >99.8%. Iron can interconvert between ferric (Fe2+) and ferrous (Fe3+) ions by accepting and donating electrons quite readily, which makes it a useful component for cytochromes, oxygen-binding molecules (e.g., hemoglobin and myoglobin), and/or enzymes.

In another embodiment, the metal (or the alloy) is at least partially coated by a protective layer. In some embodiments, the protective layer comprises one or more nonmetallic derivatives.

Biodegradable non-conductive polymers in conjunction with biodegradable conductive metal:
In an embodiment, a mesh structure of implantable device 110 may employ biocompatible, biodegradable, and/or non-conductive polymer fibers that are interwoven with biodegradable conductive metals.

Biodegradable implantable device 110 or a portion thereof can be degraded with time at a known, pre-designed rate until the completion of the healing process, thus, for example, circumventing the need to perform unnecessary surgical procedures to remove the supporting implant and significantly reduce the risks and costs involved.

The biodegradable polymer mesh (materials such as, for example, PGA and/or PLA) may have a profile of, e.g., 100 pm 1 mm in diameter, while the metal fibers may have a profile ranging, for example, from 5 to 20 pm or 5 to 500 pm.

Non-biodegradable, non-conductive polymers in conjunction with biodegradable conductive metal:
In an embodiment, a mesh structure of implantable device 110 may employ biocompatible, non-biodegradable, non-conductive polymer fibers such as, without limitation, nylon, polyethylene terephthalate (PET), ultra-high-molecular-weight polyethylene (UHMPE), etc., and may be interwoven with biodegradable conductive metal fibers. The nonbiodegradable mesh polymer can serve as a mechanical carrier for the biodegradable conductive metal.

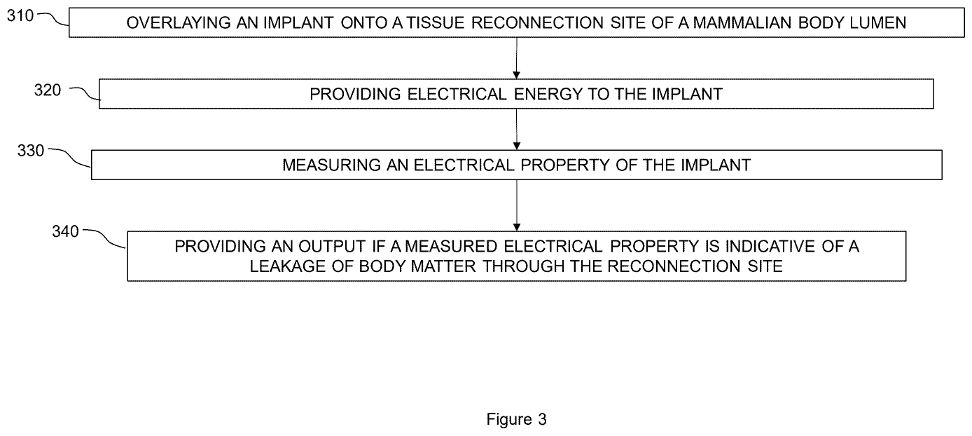

Further reference is made to FIG. 3. According to an embodiment, a method for detecting leakage of matter from a mammalian body organ may include, as indicated by box 310, overlaying implantable device 110 onto tissue reconnection site 102 of a mammalian body organ.

The method may further include, as indicated by box 320, providing electrical energy to implantable device 110.

The method may include, as indicated by box 330, measuring an electrical property of implantable device 110.

The method may further include, as indicated by box 340, providing an output if a measured electrical property is indicative of a leakage of matter through the reconnection site.

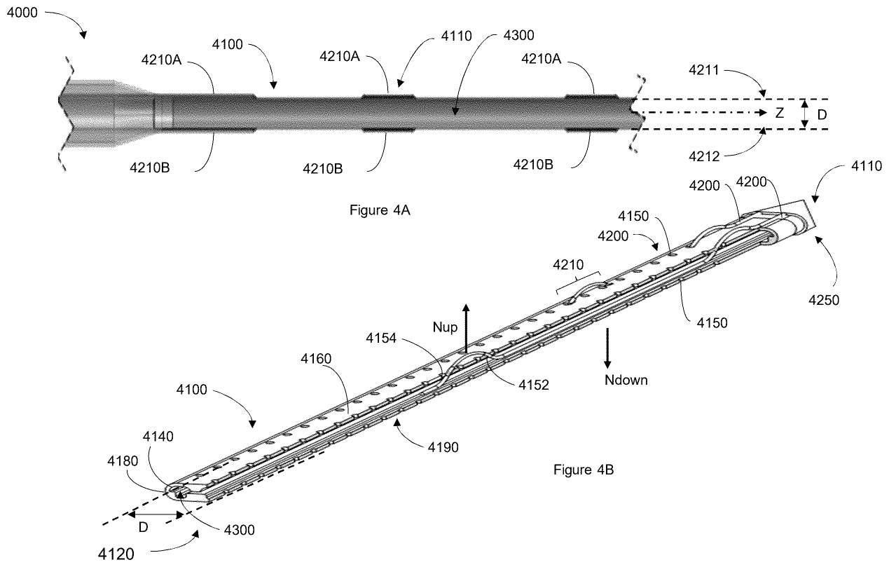

Further reference is made to FIG. 4A and FIG. 4B. In some embodiments, system 100 may include an implantable device 4000 comprising an electrode cable guide 4100 configured to accommodate one or more electrode cables 4200 to extend along a longitudinal guide axis Z from a proximal guide end 4110 to a distal guide end 4120. Implantable device 4000 may further include a drainage tube 4300 allowing drainage of bodily fluids from within the patient to outside the patient.

When operably engaged with patient tissue, a distal end of implantable device 4000 may pertain to a location that is closer to the rectum than a proximal end of implantable device 4000. In some examples, when the plurality of electrodes are operably engaged with patient tissue, “a distal location” may pertain to a location that is further away from an anastomosis site than a proximal location. Electrode cable guide 4100 may be fully biodegradable, partially biodegradable, or non-biodegradable.

An electrode cable 4200 includes a conductive wire insulated by an insulating sheath. At one or more selected portions along electrode cable 4200, the conductive wire may be non-insulated to expose a conductive wire portion 4210. The exposed conductive wire portion can engage with biological tissue to function as an electrode 4210 to measure one or more electrical characteristics of the biological tissue such as, for example, impedance.

Electrode cable guide 4100 may comprise one or more cable tracks 4140 and a plurality of guide loops 4150 arranged along guide axis Z. The plurality of guide loops 4150 may include open and/or closed guide loops, and extend from within cable tracks 4140 through a guide wall and terminate at an external surface 4160 of electrode cable guide 4100. The guide loops 4150 may be arranged to extend along guide axis Z. In some examples, a plurality of guide loops 4150 may be arranged to extend linearly to implement an (e.g., linear) cable guide path. Guide loops 4150 are configured such that electrode cables 4200 can be threaded therethrough. Additionally or alternatively, electrode cables 4200 may be weaved through, affixed, glued, stapled and/or otherwise coupled with electrode cable guide 4100.

In some embodiments, an electrode cable 4200 may extend from a connector 4250 into cable track 4140, and may further be threaded from within cable tracks 4140 via an exit guide loop 4152 to extend beyond external surface 4160. Electrode cable 4200 may further be threaded via an entry guide loop 4154 to reenter cable track 4140 and be arranged to further distally extend along electrode cable guide 4100. A distal cable end of an electrode cable 4200 may terminate within or outside cable track 4140. The cable portion extending from exit loop 4152 to entry loop 4154 may include an exposed conductive wire portion. Depending on a desired implantable device, a guide loop 4150 may function as an exit loop or as an entry loop.

In some embodiments, cable guide 4100 may be configured to accommodate at least two electrode cables 4200, which extend from connector 4250 along guide axis Z at a lateral distance D from each other, delineating respective (e.g., linear) cable guide paths. In some examples, a first set of electrodes 4210A may be arranged along a first cable guide path 4211, and a second set of electrodes 4210B may be arranged along a second cable guide path 4212, at a distance D from first cable guide path 4211. In some examples, the distance between the two or more cable guide paths may vary. Electrodes 4210 may include at least one reference electrode and at least one measurement electrode (also: intervention site electrode).

In some embodiments, leak monitoring device 120 may be configured to verify that the at least one reference electrode is operably engaged with a tissue portion that is sufficiently distant from an intervention site (e.g., at least about 5 mm, at least about 10 mm, at least about 15 mm, at least about 20 mm, at least about 30 mm, at least about 40 mm at least about 50 mm, at least about 60 mm, at least about 70 mm, or at least about 80mm), so that changes in electrical characteristics that may post-operatively occur at the intervention site, e.g., due to adverse processes and/or events, do not (significantly) influence electrical characteristics that may be measured post-operatively and e.g., concurrently, at the reference site. In some examples, acute leak may be detected using e.g., impedance measurement at the intervention site (e.g., surgical tissue connection site) by at least one measurement electrode (also: intervention electrode) and a reference site by at least one reference electrode.

In some examples, an electrode cable 4200 extending along a cable guide path may include a plurality of conductive wires that are insulated from each other. Each conductive wire may be exposed at a different position along guide axis Z, such that a corresponding plurality of electrodes 4210 may be arranged along a same guide path. For example, two or more first electrodes 4210A may be arranged along first electrode guide path 4211, and two or more second electrodes 4210B may be arranged along second electrode guide path 4212. In some examples, the two or more first electrodes 4210A of a first electrode cable 4200A may be arranged opposite the two or more second electrodes 4210B of a second electrode cable 4200B. In some examples, the two or more first electrodes 4210A may be shifted along axis Z relative to the two or more second electrodes 4210B. In some examples, at least one first electrode 4210A may be positioned opposite at least one second electrode 4210B, i.e., located at the same position along axis Z; and at least one other first electrode 4210A may be shifted along axis Z relative to at least one other second electrode 4210B. In some examples, the number of at least one first electrodes 4210A arranged along first guide path 4211 may be equal to the number of the at least one second electrodes 4210B arranged along second guide path 4212. In some examples, the number of at least one first electrodes 4210A arranged along first guide path 4211 may differ from the number of the at least one second electrodes 4210B arranged along second guide path 4212.

Once electrode cable 4200 is accommodated, wire guide 4100 may provide frictional resistance in longitudinal Z-direction, preventing electrode cable 4200 from inadvertently slipping within electrode cable guide 4100. This way, the conductive electrode portions 4210 remain exposed, ensuring contact of the electrode portions with surrounding biological tissue.

In some examples, as shown schematically in FIG. 4A, electrode cable 4200 may be accommodated such that an exposed conductive wire portion 4210 is in direct contact with external surface 4160 of cable guide 4100, before implantable device 4000 is operably engaged with a patient SOI to be monitored by the implantable device.

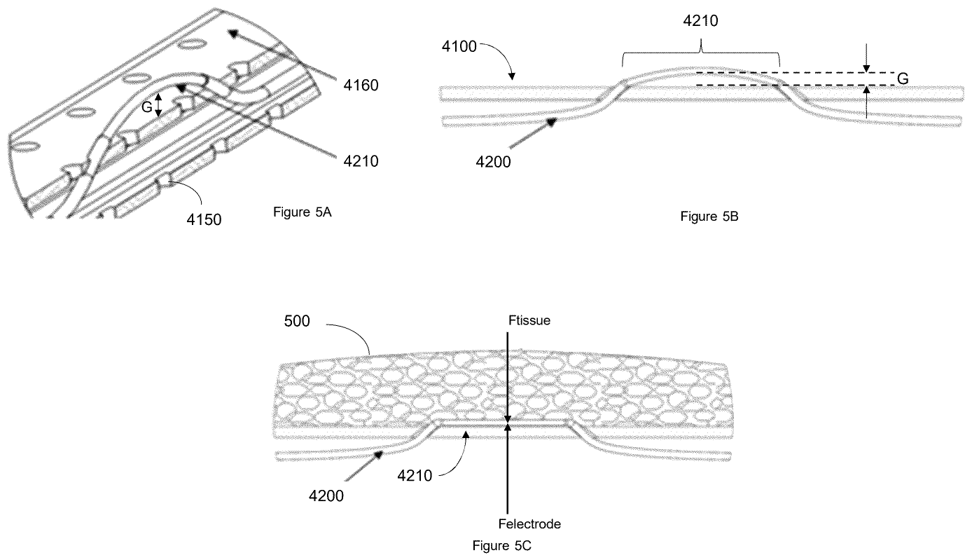

Additional reference is made to FIGS. 5A-5C. In some embodiments, before implantable device 4000 is operably engaged with a patient SOI (e.g., biological tissue) 500, an exposed conductive wire portion 4210, which may constitute an electrode, may have a bent or curved configuration, as schematically shown in FIG. 5A and FIG. 5B. Electrode 4210 may be bent away or upwards in direction Nup relative to an underlying upper guide surface 4160, forming a gap G therebetween.

Operably engaging implantable device 4000 with a patient SOI for monitoring tissue 500, causes electrode 4210 to become sandwiched between tissue 500 and the external electrode guide surface 4160. Since prior to operable engagement, electrode 4210 has a convex configuration with respect to the underlying external surface 4160, operably engaging electrode 4210 with tissue 500 may result in that that the electrode-engaging portion of tissue 500 applies a counterforce Ftissue against upward bent electrode 4210, causing electrode 4210 to be pressed against underlying external surface 4160, e.g., as shown schematically in FIG. 5C. Correspondingly, electrode 4210 presses against tissue 500 with a force Felectrode=Ftissue. The pre-bent portion of electrode 4210 may thus result in increasing the contact force Felectrode applied by electrode 4210 onto tissue 500, compared to the contact force that would be applied by electrode 4210 onto tissue 500 if electrode 4210 had not a pre-bent configuration, e.g., as schematically shown in FIG. 4A.

In some embodiments, leak monitoring device 120 (cf. FIG. 1) may be operably coupled with implantable device 4000 to provide a user with a clinically interpretable output relating to physical quantities sensed by electrodes 4210 accommodated by electrode guide 4100.

In some examples, leak monitoring device 120 may be configured to determine, based on signals provided by one or more electrodes 4210, whether implantable device 4000 properly or improperly engages with a patient SOI. Improper engagement (also: inoperable engagement of implantable device 4000 with the patient) may prevent leak monitoring device 120 from providing a clinically interpretable output respective of the one or more electrodes 4210.

In some embodiments, leak monitoring device 120 may be configured to detect improper engagement of implantable device 4000 with a patient SOI (e.g., wrong placement, displaced, slippage, detachment, and/or the like, relative to tissue 500), for instance, by determining whether a signal produced by an electrode meets an “electrode problem criterion”, which may relate to one or more thresholds of electrical parameter values. An electrode problem criterion may be met if a signal output exceeds a high threshold value (e.g., exceeds a high impedance threshold value), and/or drops below a low threshold value (e.g., drops below an electrical current threshold value). For example, if an impedance value produced with respect to an electrode exceeds, e.g., 20-100 kOhm, leak monitoring device 120 may provide an output relating to a “electrode misplacement alert”, e.g., for the corresponding electrode 4210, and/or implantable device 4000. In case of a misplacement, instructions would be provided to the user to properly position electrode guide 4100 to improve the positioning of electrodes 4210 with the patient SOI. Alternatively or additionally, measurement (also: sensing) of electrical characteristics while performing a medical procedure (e.g., surgery) at the patient SOI, and/or postoperatively, may be used by the system to indicate tissue properties such as, for example, perfusion, inflammation, necrosis, and/or fibrosis.

In some embodiments, leak monitoring device 120 may be configured to determine whether an anomaly pertaining to electrode output pertains to or more likely pertains to either electrode displacement or hardware and/or software system 100 malfunction. Leak monitoring device 120 may be configured to compare the outputs of one or more first electrodes against the outputs produced by one or more second electrodes. For example, if electronic signals that are output by two or more (e.g., neighboring) electrodes 4210 meet the “electrode problem criterion”, e.g., exceed a high impedance threshold, system 100 may derive that the anomaly is caused rather or more likely due to displacement of implantable device 4000 relative to the patient SOI than due to electrode malfunction. On the other hand, if for instance only the signal output received from only one of plurality of electrodes meets the “electrode problem criterion”, it may be determined that the anomaly is caused rather due to malfunction of the respective electrode. In such scenario, device 120 may provide an output indicating which electrode is (likely) malfunctioning.

In some embodiment, electrode guide 4100 may be configured from an expanded (e.g., unfolded) to a collapsed (e.g., folded) configuration. In the unfolded configuration, cable track 4140 of implantable device 4000 may be exposed or unfolded, facilitating accommodating electrode cables 4200 in the exposed part of cable track 4140 and facilitate threading and/or pushing electrode cables 4200 in loops 4150 such that conductive wire portions 4210 can operably engage with a patient SOI when electrode guide 4100 is brought into the collapsed configuration. In some examples, electrode guide 4100 may have a folding line 4180 longitudinally extending along guide axis Z.

In some examples, electrode guide 4100 may be made of a flexible material, suitable for use within a patient. Electrode guide 4100 may be sufficiently flexible to conform with the contour of a patient SOI.

In some embodiments, electrodes 4210 may be accommodated on either or both sides of an electrode guide 4100, which may for example have a flat strip-like geometry. For example, electrode cables 4200 may be accommodated by electrode guide 4100 such that at least one of a plurality of electrodes 4210 is arranged to extend over upper guide surface 4160, e.g., show schematically in FIG. 4B, and further such that at least one other electrode (not shown) is arranged to extend over lower guide surface 4190. In some examples, the at least one other electrode (not shown) may have a bent configuration, i.e., bent downwards away from lower guide surface 4190.

In some examples, implantable device 4000 may be configured and/or electrodes 4210 may be accommodated such at least one first electrode can operably engage with tissue of a first organ comprising the patient SOI, and at least one second electrode can concurrently operably engage with a second organ that is different from the first organ. In some embodiments, electrodes 4210 may be arranged, e.g., about opposite each other of guide 4100 (e.g., cf. FIG. 4A).

Based on signals received from opposite arranged electrodes 4210A and electrodes 4210B, system 100 may identify which electrodes are in operable contact with the patient SOI to be monitored, for example, based on analyzing noise and/or signal characteristics of the received output signals.

Positional terms such as “upper”, “lower” “right”, “left”, “bottom”, “below”, “lowered”, “low”, “top”, “above”, “elevated”, “high”, “vertical” and “horizontal” as well as grammatical variations thereof as may be used herein do not necessarily indicate that, for example, a “bottom” component is below a “top” component, or that a component that is “below” is indeed “below” another component or that a component that is “above” is indeed “above” another component as such directions, components or both may be flipped, rotated, moved in space, placed in a diagonal orientation or position, placed horizontally or vertically, or similarly modified. Accordingly, it will be appreciated that the terms “bottom”, “below”, “top” and “above” may be used herein for exemplary purposes only, to illustrate the relative positioning or placement of certain components, to indicate a first and a second component or to do both.

In some embodiments, an electrode guide may have various cross-sectional geometries when in the collapsed or reduce (e.g., folded) configuration. Accordingly, the electrode guides discussed may not be limited to the geometry shown for instance in FIG. 4B. In some examples, when in the reduced or folded configuration, an electrode guide may have an about circular, oval, polygonal (e.g., triangular) cross-section, when viewed along the guide axis Z. In some examples, an electrode guide having a polygonal geometry may accommodate electrode cables such that electrodes 4210 may extend along each side of the electrode guide so that the accommodated electrodes are facing outwards external to corresponding outer guide surfaces.

In some examples, an electrode 4210 may extend into drainage tube 4300, e.g., for measuring an electric characteristic relating to bodily fluids drained through drainage lumen 4300 (not shown).

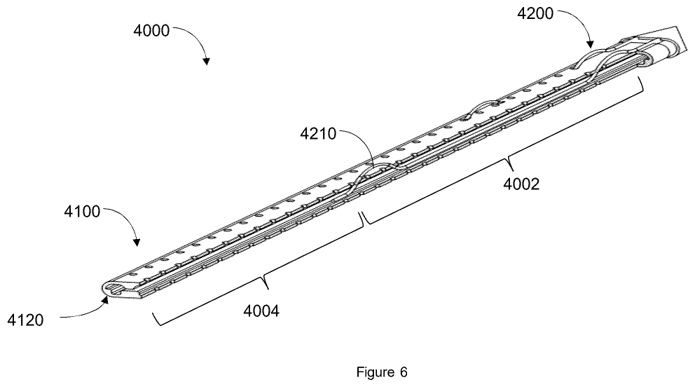

Further reference is made to FIG. 6. In some embodiments, implantable device 4000 may comprise a measurement section 4002 comprising electrodes 4210, and a non-measurement section 4004 that is free of electrodes 4210. In some examples, a distal portion of electrode guide 4100 may be electrode-free (“non-measurement section 4004”), and a proximal portion of electrode guide 4100 may comprise electrodes (“measurement section 4002”). Measurement section 4002 and/or non-measurement section 4004 may be flexible or bendable. In some examples, one of measurement section 4002 and/or non-measurement section 4004 may be comparatively stiff, i.e., non-bendable. Configuring implantable device 4000 to have a measurement-free section 4004 may facilitate operably engaging implantable device 4000 with a patient SOI, without requiring touching of electrodes by the medical professional or robot during the placement procedure (e.g., intraoperatively). Measurement-free section 4004 may be used as guide for ensuring correct placement the sensing electrode and the reference electrode with the patient SOI, even if deployed in a blind manner. For instance, correct placement (also: operable engagement) of implantable device 4000 may be ensured based on electrode measurement outputs. For example, system 100 may provide, based on the electrode measurements, an output indicative of correct or incorrect placement of implantable device 4000 with the patient SOI. The output may be provided to a medical professional a visual output, an auditory output, a tactile output, and/or the like. In some examples, the output may additionally or alternatively be an electronic signal provided to a robot employed for placing the implantable device 4000.

In some embodiments, implantable device 4000 may be deployed such that it is sandwiched between two tissue portions, of which is one tissue pertains to the patient SOI, for monitoring the patient SOI and determining, based on sensed electrical parameter values of the patient SOI, whether the patient SOI is experiencing an adverse event, process and/or condition (e.g., leak, inflammation, etc.). In some examples, implantable device 4000 may be coupled with a patient SOI using a fastener such as, for example, a suture, a glue, staplers, and/or the like.

In some embodiments, system 100 may determine a probability that the patient will experience an adverse post-operative event with respect to the SOI. In some embodiments, system 100 may determine an expected onset time at which a patient starts to experience a clinically adverse event with respect to a patient SOI (e.g., leakage onset time, tissue inflammation onset time, etc.).

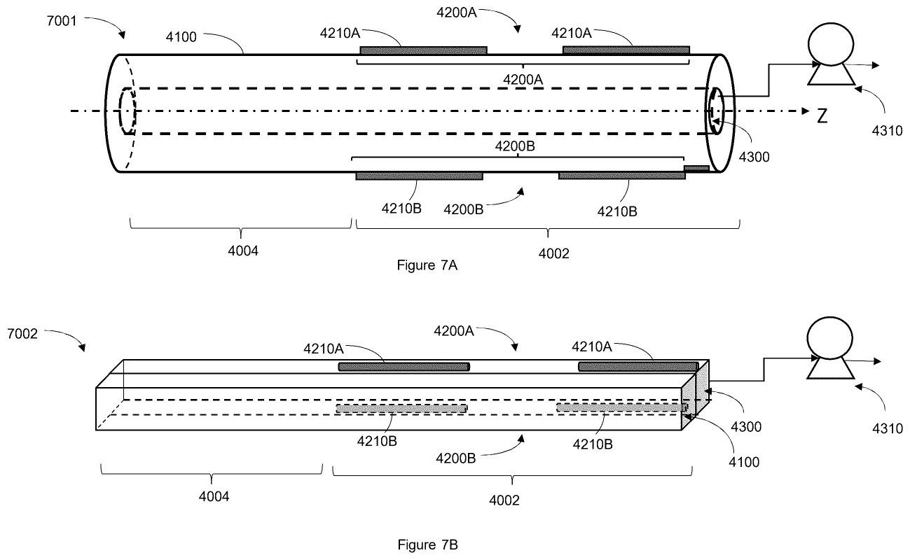

Additional reference is made to FIG. 7A. In some embodiments, an implantable device 7001 may be configured to have a drainage 4300 that is fluidly sealed or insulated from electrode cable guide 4100. For instance, drainage 4300 and cable guide 4100 may be coaxially arranged. Drainage 4300 may be interior to cable guide 4100, so that that electrodes 4210 can make directly operably engaged with a patient SOI exterior to implantable device 7001. While cable guide 4100 and drainage 4300 are shown to have circular cross-sections, these should by no means be construed in a limiting. Accordingly, a coaxial arrangement of an outer cable guide 4100 and inner drainage 4300 may each have varying or alternative geometric cross-sections.

Further reference is made to FIG. 7B. In some embodiments, an implantable device 7002 comprises a cable guide 4100 configured to guide electrode cables 4200, and a drainage tube 4300 that may be fluidly insulated with respect to and juxtaposed of cable guide 4100. While in FIG. 7B cable guide 4100 and drainage 4300 are each shown to have rectangular cross-sections, these should by no means be construed in a limiting. Accordingly, a coaxial arrangement of an outer cable guide 4100 and inner drainage 4300 may each have varying or alternative geometric cross-sections.

In some embodiments, bodily fluid contained in drainage tube 4300 may be drained through gravitation, or by employing a drainage suction pump 4310 that is be fluidly coupled with drainage tube 4300. In some examples, drained bodily fluid may be analyzed by system 100, e.g., for determining whether an adverse event has occurred and/or is about to occur with the respect to the patient SOI. The analysis result of the bodily fluid may be compared with analysis results of electronic signals produced by electrodes 4210. That drainage suction pump 4310 may be a manually operatable pump, or an automatically operatable pump. In some embodiments, suction pump 4310 may be an expandable suction balloon. The terms “suction” and “aspiration” may herein be used interchangeably.

As mentioned herein, electrodes cables 4200 may be arranged to extend along main longitudinal axis Z of electrode cable guide 4100. Applying a pulling force in direction of axis Z to remove implantable device 4000 causes electrodes 4210 to be pulled out about in direction of the electrodes'longitudinal orientation. This way, the risk of inflicting injury to surrounding tissue 500 due to removal of the electrodes may be reduced or minimized, compared to the risk of inflicting injury to tissue if the electrodes were arranged about orthogonally relative to a pulling or removal direction.

Additional Examples

Example 1 pertains to an implantable device for monitoring a patient site of interest (SOI) of a mammalian subject, the device comprising:
an electrode cable guide extending along a longitudinal direction;
the guide comprising having an outer guide surface:
   one or more electrode cable tracks extending along the longitudinal direction;
a plurality of loops arranged to extend along the longitudinal direction;
wherein the plurality of loops and the tracks are configured to accommodate at least one electrode cable having at least two non-insulated conductive wire portions such that the conductive wire portions are arranged on the outer surface for engaging with a tissue site of interest of a mammalian subject; and
to accommodate electrically insulated cable portions within the cable tracks underneath the outer guide surface.

Example 2 includes the subject matter of example 1 and, optionally, wherein the electrode cable comprises two electrodes separated from each other so that the two electrodes can be operably engaged with the patient SOI for providing an output relating to an electrical parameter value of the patient SOI.

Example 3 includes the subject matter of example 2 and/or example 2 and, optionally, a drainage tube configured to drain bodily fluid from the patient SOI to outside the patient.

Example 4 includes the subject matter of any one or more of the examples 1 to 3 and, optionally, configured to accommodate at least one measurement electrode and at least one reference electrode.

Example 5 includes the subject matter of any one or more of the examples 1 to 4 and, optionally, configured such that accommodated electrode cables extend along the longitudinal device axis.

Example 6 includes the subject matter of any one or more of the examples 1 to 5 and, optionally, a section that is free of measurement and/or reference electrodes.

Example 7 includes the subject matter of any one or more of the examples 1 to 6 and, optionally, having a longitudinal strip-like configuration.

Example 8 pertains to a system for monitoring a patient site of interest (SOI) of a mammalian subject, the system comprising:
an implantable device configured to accommodate at least one measurement electrode and at least one reference electrode for operably engagement with a patient SOI;
a memory; and
a processor; configured to perform the following:
   receiving electrical signals received from the at least one measurement electrode and the at least one reference measurement electrode;
processing the received electrode signals; and
detecting, based on the processing of the received electrode signals, an anomaly with respect to an electrical characteristic of the patient SOI.

Example 9 includes the subject matter of example 8 and, optionally, further configured to distinguish between an anomaly that pertains to the patient SOI and an anomaly that pertains to system malfunction.

Example 10 pertains to a method of monitoring a patient site of interest (SOI) of a mammalian subject, the method comprising:
placing an electrode cable guide extending along a longitudinal direction;
the guide comprising having an outer guide surface:
   one or more electrode cable tracks extending along the longitudinal direction;
a plurality of loops arranged to extend along the longitudinal direction;
wherein the plurality of loops and the tracks are configured to accommodate at least one electrode cable having at least two non-insulated conductive wire portions such that the conductive wire portions are arranged on the outer surface for engaging with a tissue site of interest of a mammalian subject; and
to accommodate electrically insulated cable portions within the cable tracks underneath the outer guide surface.

Example 11 pertains to a method for monitoring a patient site of interest (SOI) of a mammalian subject, the method comprising:
receiving electrical signals received from the at least one measurement electrode and the at least one reference measurement electrode;
processing the received electrode signals; and
detecting, based on the processing of the received electrode signals, an anomaly with respect to an electrical characteristic of the patient SOI.

Example 12 includes the subject matter of example 11 and, optionally, distinguishing between an anomaly that pertains to the patient SOI and an anomaly that pertains to system malfunction.

The various features and steps discussed above, as well as other known equivalents for each such feature or step, can be mixed and matched by one of ordinary skill in this art to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein. For example, any digital computer system can be configured or otherwise programmed to implement a method disclosed herein, and to the extent that a particular digital computer system is configured to implement such a method, it is within the scope and spirit of the disclosure. Once a digital computer system is programmed to perform particular functions pursuant to computer-executable instructions from program software that implements a method disclosed herein, it in effect becomes a special purpose computer particular to an embodiment of the method disclosed herein. The techniques necessary to achieve this are well known to those skilled in the art and thus are not further described herein. The methods and/or processes disclosed herein may be implemented as a computer program product such as, for example, a computer program tangibly embodied in an information canier, for example, in a non-transitory computer-readable or non-transitory machine-readable storage device and/or in a propagated signal, for execution by or to control the operation of, a data processing apparatus including, for example, one or more programmable processors and/or one or more computers. The terms “non-transitory computerreadable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by a communication network.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

“Coupled with” means indirectly or directly “coupled with”.

Where applicable, although state diagrams, flow diagrams or both may be used to describe embodiments, the technique is not limited to those diagrams or to the corresponding descriptions. For example, flow need not move through each illustrated box or state, or in exactly the same order as illustrated and described.

It should be understood that where the claims or specification refer to “a” or “an” element, such reference is not to be construed as there being only one of that element.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made.

Unless otherwise specified, the terms “substantially”, “about” and/or “close” with respect to a magnitude or a numerical value may imply to be within an inclusive range of −10% to +10% of the respective magnitude or value.

“Coupled with” can mean indirectly or directly “coupled with”.

It is important to note that the method may include is not limited to those diagrams or to the corresponding descriptions. For example, the method may include additional or even fewer processes or operations in comparison to what is described in the figures. In addition, embodiments of the method are not necessarily limited to the chronological order as illustrated and described herein.

Discussions herein utilizing terms such as, for example, “processing”, “computing”, “calculating”, “determining”, “establishing”, “analyzing”, “checking”, “estimating”, “deriving”, “selecting”, “inferring” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes. The term determining may, where applicable, also refer to “heuristically determining”.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not be construed as excluding an embodiment referring to a condition “equal or below a threshold”. It is clear that should a condition be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

It should be understood that where the claims or specification refer to “a” or “an” element and/or feature, such reference is not to be construed as there being only one of that element. Hence, reference to “an element” or “at least one element” for instance may also encompass “one or more elements”.

Terms used in the singular shall also include the plural, except where expressly otherwise stated or where the context otherwise requires.

In the description and claims of the present application, each of the verbs, “comprise” “include” and “have”, and conjugates thereof, are used to indicate that the data portion or data portions of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made. Further, the use of the expression “and/or” may be used interchangeably with the expressions “at least one of the following”, “any one of the following” or “one or more of the following”, followed by a listing of the various options.

As used herein, the phrase “A, B, C, or any combination of the aforesaid” should be interpreted as meaning all of the following: (i) A or B or C or any combination of A, B, and C, (ii) at least one of A, B, and C; (iii) A, and/or B and/or C, and (iv) A, B and/or C. Where appropriate, the phrase A, B and/or C can be interpreted as meaning A, B or C. The phrase A, B or C should be interpreted as meaning “selected from the group consisting of A, B and C”. This concept is illustrated for three elements (i.e., A, B, C), but extends to fewer and greater numbers of elements (e.g., A, B, C, D, etc.).

It is appreciated that certain features of the invention, which are, for clarity, described in the context of separate embodiments and/or example, may also be provided in combination in a single embodiment. Conversely, various features of the invention, which are, for brevity, described in the context of a single embodiment, example and/or option, may also be provided separately or in any suitable sub-combination or as suitable in any other described embodiment, example or option of the invention. Certain features described in the context of various embodiments, examples and/or optional implementation are not to be considered essential features of those embodiments, unless the embodiment, example and/or optional implementation is inoperative without those elements.

It is noted that the terms “in some embodiments”, “according to some embodiments”, “for example”, “e.g.”, “for instance” and “optionally” may herein be used interchangeably.

The number of elements shown in the Figures should by no means be construed as limiting and is for illustrative purposes only.

“Real-time” as used herein generally refers to the updating of information at essentially the same rate as the data is received. More specifically, in the context of the present invention “real-time” is intended to mean that the image data is acquired, processed, and transmitted from a sensor at a high enough data rate and at a low enough time delay that when the data is displayed, data portions presented and/or displayed in the visualization move smoothly without user-noticeable judder, latency or lag.

It is noted that the terms “operable to” can encompass the meaning of the term “modified or configured to”. In other words, a machine “operable to” perform a task can in some embodiments, embrace a mere capability (e.g., “modified”) to perform the function and, in some other embodiments, a machine that is actually made (e.g., “configured”) to perform the function.

Throughout this application, various embodiments may be presented in and/or relate to a range format. It should be understood that the description in range format is merely for convenience and brevity and should not be construed as an inflexible limitation on the scope of the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

### EXAMPLES

Reference is now made to the following examples, which together with the above descriptions illustrate some embodiments of the invention in a non-limiting fashion.

### Example 1

### General Procedure

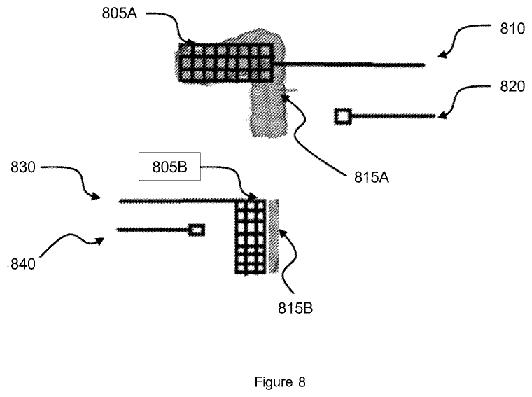

Reference is now made to FIG. 8, which illustrates a colon as the surgical site 815A and a mesh 805A being implanted thereon.

In exemplary procedures, two meshes (detection electrode, and reference electrode) in a mesh density of 1 to 8 mm×1 to 8 mm and at least 2 mm×2 mm opening composed of wires (d=150 pm) are implanted, one (805A) at the surgical site (815A) and another site (reference site 805B) at a different location of the colon (815B) further from the surgical site.

In exemplary procedures, potential or current are measured between surgical site, points 810, and/or 820 and references sites, 830, and/or 840 and are compared.

In exemplary procedures, an inflammatory response occurs owing to GI leakage that triggers inflammatory cascade which in turn provides high corrosive environment at the surgical site.

In exemplary procedures, the mesh at the surgical site degrades faster compared to the reference mesh electrode. The potential (or current) measured between points 810, 820 is higher compared to the potential (or current) measured between points 830, 840.

In exemplary procedures, the potential (or current) is measured right after surgery and 2, 3, 4 to 6 days post-surgery.

In additional exemplary procedures, the two electrodes are placed at the same site during surgery, whereas one electrode known to be less influenced by inflammatory response and its degradation would be relatively stable.

In exemplary procedures, an electrode known to be less influenced by inflammatory response is obtained by its coating thereof with slow (about a year) degradable polymer, or other type of metal/alloy.

In additional exemplary procedures alternating current (AC) electrochemical impedance is measured.

In additional exemplary procedures, the coating is 10-100 pm thickness.

In additional exemplary procedures, the coating is permeable to the electrolyte solution so as to allow current/potential measurement in relative to the specific body fluids.

### Example 2

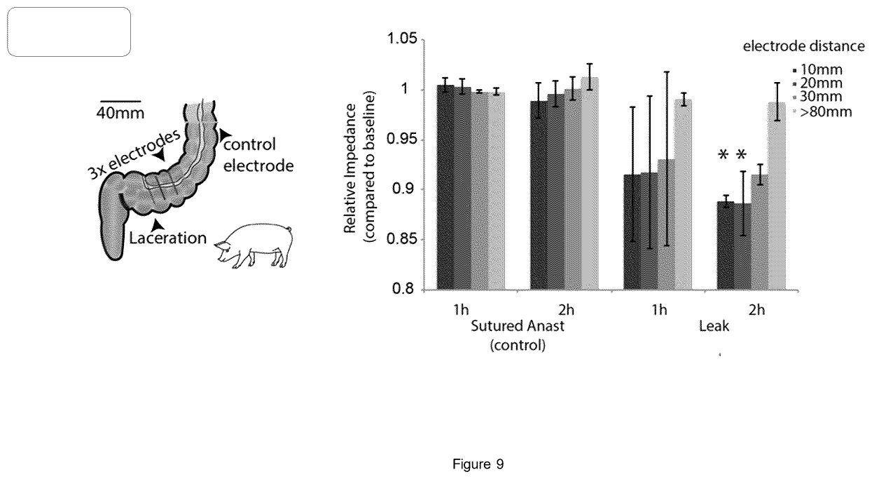

As shown schematically in FIG. 9, acute leak was detected using impedance measurement at the surgical site and a distant reference electrode (placed about 80 mm and more, from the site, sensing the same tissue or organ). Compared to the reference site (>80 mm), tissue impedance decreased by about 10% within 10-30 mm from the leak site. The drop in impedance enables detection of leak starting 2 hours after leak induction.

What is claimed is:

1. An implantable device for monitoring a patient site of interest (SOI) of a mammalian subject, the device comprising:
an electrode cable guide extending along a longitudinal direction;
the guide comprising having an outer guide surface:
one or more electrode cable tracks extending along the longitudinal direction;
a plurality of loops arranged to extend along the longitudinal direction;
wherein the plurality of loops and the tracks are configured to accommodate at least one electrode cable having at least two non-insulated conductive wire portions such that the conductive wire portions are arranged on the outer surface for engaging with a tissue site of interest of a mammalian subject; and
to accommodate electrically insulated cable portions within the cable tracks underneath the outer guide surface.

2. The implantable device of claim 1, wherein the electrode cable comprises two electrodes separated from each other so that the two electrodes can be operably engaged with the patient SOI for providing an output relating to an electrical parameter value of the patient SOI.

3. The implantable device of claim 2, further comprising:
a drainage tube configured to drain bodily fluid from the patient SOI to outside the patient.

4. The implantable device of claim 1, configured to accommodate at least one measurement electrode and at least one reference electrode.

5. The implantable device of claim 1, configured such that accommodated electrode cables extend along the longitudinal device axis.

6. The implantable device of claim 1, comprising a section that is free of measurement and/or reference electrodes.

7. The implantable device of claim 1, having a longitudinal strip-like configuration.

8. A system for monitoring a patient site of interest (SOI) of a mammalian subject, the system comprising:
an implantable device configured to accommodate at least one measurement electrode and at least one reference electrode for operably engagement with a patient SOI;
a memory; and
a processor; configured to perform the following:
receiving electrical signals received from the at least one measurement electrode and the at least one reference measurement electrode;
processing the received electrode signals; and
detecting, based on the processing of the received electrode signals, an anomaly with respect to an electrical characteristic of the patient SOI.

9. The system of claim 8, further configured to distinguish between an anomaly that pertains to the patient SOI and an anomaly that pertains to system malfunction.

10. (canceled)

11. A method for monitoring a patient site of interest (SOI) of a mammalian subject, the method comprising:
receiving electrical signals received from the at least one measurement electrode and the at least one reference measurement electrode;
processing the received electrode signals; and
detecting, based on the processing of the received electrode signals, an anomaly with respect to an electrical characteristic of the patient SOI.

12. The method of claim 11, further comprising distinguishing between an anomaly that pertains to the patient SOI and an anomaly that pertains to system malfunction.

13. The implantable device of claim 1, wherein prior to operable engagement with the SOI, the conductive wire portions have a convex configuration with respect to the underlying tissue.

14. The implantable device of claim 1, wherein the at least one electrode cable is threaded through the loops of the electrode cable guide.

15. The implantable device of claim 1, wherein the electrode cable guide is configurable from an expanded configuration to a collapsed configuration, and implantable device of claim 1, wherein in the expanded configuration that cable track is exposed to facilitate accommodating the electrode cables.

16. The implantable device of claim 1, wherein the electrode cable guide comprises a folding line.

17. The implantable device of claim 1, wherein the electrode cable guide has a flat strip-like configuration.

18. The implantable device of claim 1, wherein the electrode cable guide is configured to allow accommodating at least one electrode either or both sides of the electrode cable guide.

19. The implantable device of claim 1, wherein the electrode cable guide is sufficiently flexible to conform with the contour of a patient SOI.

20. The implantable device of claim 1, comprising a connector for connecting the at least one electrode.
